(19) United States
(12) Patent Application Publication
(10) Pub. No.: US 2005/0048566 A1
(43) Pub. Date: Mar. 3, 2005

(54) Apparatus, composition and method for proteome profiling

(76) Inventors: Charles Delisi, Brookline, MA (US); Richard Laursen, Newton, MA (US); Zhiping Weng, Roslindale, MA (US); Adnan Derti, Brighton, MA (US); Sergei Ivanov, Newton, MA (US); Andre Sharon, Newton, MA (US)

Correspondence Address: Ronald I Eisenstein;Nixon Peabody, 101 Federal Street, Boston, MA, 02110, US

(21) Appl. No.: 10/487,919

(22) PCT Filed: Aug. 27, 2002
(86) PCT No.: PCT/US02/27261

Related U.S. Application Data
(60) Provisional application No. 60/315,157, filed on Aug. 27, 2001.

(51) Int. Cl. G01N 33/53; G01N 33/543

(52) U.S. Cl. 435/7.1; 436/518

(57) ABSTRACT

The present invention is directed to a high throughput method for producing a large number of different antibodies, more specifically organized antibody microarrays. These antibodies and antibody microarrays can be used to rapidly assay protein abundance and identify types of proteins that are expressed in cells and tissues under a variety of conditions, or to compare protein expression profiles of different cells.

### FIELD OF THE INVENTION

The present invention is directed to a method for rapid determination of proteins expressed by a particular cell of a known genome and the apparatus which permits such determination. For example, this method can be used to determine which proteins are differentially expressed in a malignant cell when compared to a wild type cell.

### BACKGROUND OF THE INVENTION

Significant attention in recent years has been directed to understanding and categorizing the genome of various organisms including humans. That field has been referred to as genomics.

Attention has also been focused on understanding and identifying the various proteins an organism expresses. This field is referred to as proteomics. Comparisons of genes expressed by various organisms show greater similarity than might be expected by the physical differences between the species. Thus, understanding the proteins that are expressed, when they are expressed, and in what cells they are expressed takes on increasing importance.

This is also important with respect to diseases, malignancies, etc. Consequently, ascertaining the set of proteins expressed by a particular cell type at various times and states such as resting vs. developing, normal (wild type), malignant, diseased, etc. has been an important challenge. Any method that could even partially meet this challenge, for example by determining a fraction of the protein profile rapidly and cost effectively, would be extremely desirable.

The typical approach used in assessing the number and identity of expressed proteins is 2D gel electrophoresis and its extensions. The method, which was introduced 25 years ago, separates proteins on the basis of size and charge, and typically resolves several thousand proteins (1). More recently, mass spectrometry (MS) has been used in conjunction with the 2D gels after proteolytic cleavage to quantitatively ascertain the mass associated with each spot and to help identify the protein. However, these methods have various drawbacks.

Among the problems associated with the use of gels and MS are preparation and purification of proteins, resolution and throughput. Although MS solves some of the problem of spot identification, its application to large numbers of spots (100 or more) is slow. Other problems are limitations in dynamic range of abundance and mass, For example, proteins expressed in low amounts are frequently missed. Further, the use of denaturants can prevent related functional studies.

Ciphergen Biosystems Inc., has reported a chip technology that it claims should allow researchers to capture, separate and quantitatively analyze proteins directly on the chip. Their system is said to integrate mass spectrometry (particularly, surface enhanced laser desorption/ionization (SELDI)) and biochip technology on a single chip. They claim that their ProteinChip™ uses various molecular substrates, including antibodies and receptors, having affinities for proteins of interest. The chips are stated to be made of aluminum, about three inches long and one centimeter wide, containing eight sites and a group of 12 is alleged to be processed as the equivalent of a 96-well format. This system is intended to measure the mass of the captured proteins rather than their activity. The system is also limited in the number of kinds of proteins that can be identified. Therefore, it is not broadly applicable.

Zyomyx Inc. and CombiMatrix Corp., both California companies, have stated that they are working on creating large-scale standardized methods for producing protein biochips. Zyomyx Inc., has claimed to develop a biochip, covered with a multi-component organic thin film to reduce non-specific protein binding and a protein capture agent such as an antibody or a peptide to fish for specific proteins of interest. The binding of proteins to capture agents is said to be detected by fluorescence among other methods. However, Zyomyx's technology is concerned with immobilizing a correctly oriented protein on a solid surface which is a complex and expensive process.

CombiMatrix Corp., has reported it is developing a method, utilizing electrochemistry and semiconductor technology, to synthesize peptides (one amino acid at a time), antibodies, and proteins directly on the chip. The chip is said to consist of a large number of virtual flasks (up to one million per square centimeter) arranged in a grid pattern on the surface of a semiconductor wafer. This, too, is a very complex and expensive process.

MacBeath et al. of Harvard University have described a method of immobilizing proteins by covalently attaching them to glass surfaces that is stated as using standard laboratory equipment. MacBeath et al. reports that they were able to create protein microarrays (with about 10,800 spots per standard microscope slide). These microarrays were alleged to be effective in detecting interactions between one protein and another that are known to interact with a small molecule (for which specific protein receptors are available) and a protein, and an enzyme and its substrate by identifying phosphorylation by means of phosographic emulsion and a light microscope.

Genomic Solutions has stated it is developing robots to prepare samples (protein digestion) and to excise spots for MS. However, such a method is expensive and technologically complex.

Accordingly, a need exists for a method of determining proteins expressed by a particular cell that is relatively simple. It would be desirable if this method was fast. It would be more desirable if the method was simple.

### SUMMARY OF THE INVENTION

We have here discovered a high throughput method for producing a large number of different antibodies. These antibodies can be used to rapidly assay protein abundance in cells under a variety of conditions or to compare protein expression profiles of different cells.

Additionally, we have discovered a method for the determination of proteins expressed by a specific cell or tissue. In one embodiment, the present invention permits targets of such proteins to be obtained.

Still another embodiment of the present invention is directed to a method of making a microarray that can be used in such a method. The method of making a microarray utilizes microarrays of peptides, wherein one or more of the peptides are from a coding region of a genome of interest. Preferably, the peptides cover at least a part of the coding region of the genes that are of interest. For example, peptides can be selected from a family of proteins such as chemokine receptors, G-coupled protein receptors, a family of related proteins such as tumor associated antigens, oncogene products, etc. or combinations thereof. Preferably, the peptides chosen contain an antigenic epitope. More preferably, the peptide has an epitope that approximates the wild type conformation of the protein.

The arrays are used to screen an antibody library such as a large, combinatorially generated library of antibodies that specifically bind to the peptides. Preferably, the antibodies bind to the peptides in a conformation that approximates their native state (i.e. when they are part of the protein). In this way a large library of antibodies that will bind specific native proteins is obtained. These antibodies can be for any species whose coding genome is known for any desired group of proteins. The antibodies can then be expressed by known means such as simple bacterial amplification. The antibodies are arrayed on a substrate such as on a chip or sphere. Any type of substrate will be a suitable “chip” as long as the antibodies can be substantially immobilized and used as bait to fish for expressed proteins in a sample, such as a cell of interest. Such antibody arrays can be used to screen a biological sample of interest. The proteins in the sample that bind to the array can readily be determined.

These arrays can be used for a wide range of purposes. For example, to determine proteins that are differentially expressed in different cells. For instance, malignant cells versus non-malignant cells, diseased cells versus normal, cells in a pregnant woman versus non-pregnant, menopausal versus non-menopausal, stem cells versus nerve cells, etc. The antibody array of the present invention can be used, for example, in the diagnosis and treatment of a cancer, and immunopathology, a neuropathology, and the like.

In another aspect, the present invention provides an expression profile that can reflect the expression levels of a plurality of proteins in a sample. The expression profile comprises an antibody array and a plurality of detectable proteins.

The profiles can be collected, for example, to a database which can consequently be used for diagnostic and prognostic purposes, and for “pharmacoproteomic” applications. Such diagnostic and prognostic purposes include, for example, classification of different types of cancers according to their protein expression profile. Pharmacoproteomic applications include, for example, classification of individuals according to their responsiveness to pharmaceuticals or propensity to harmful side effects according to their protein expression profiles.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention. In the drawings,

FIG. 1 is a schematic of the automated oligonucleotide microarray fabricator. A collimated beam of UV light is shown upon the micromirror array and computer-selected micromirrors reflect the light through the projection system on to the peptide array slide, which is mounted in a flow cell. Reagents are pumped through the cell from an oligopeptide synthesizer.

FIG. 2 is an expanded schematic view of the microarray fabricator flow cell. In use, the components are clamped together and the assembly is mounted at 90° to the direction shown, with the reagents from the peptide synthesizer introduced at the bottom. This design permits UV irradiation either from the front (shown), or back of the slide.

FIG. 3 is a derivatization and synthesis of peptides on a glass surface. The linker, Nvocaminocaproic acid, is added in step 2. Abbreviations: HOBT, hydroxybenzotriazole; NMM, N-methylmorpholine; TBTU, 0-(7benzotriazol-1-yl)-1, 1,3,3-tetrametyluronium tetrafluoroborate; Nvoc, N-α-6-nitroveratyloxycarbonyl photolabile protecting group.

FIG. 4 shows how coding regions for immunoglobulins (Ig) heavy and light chain amino terminal domains are linked to form a single chain, and inserted proximal to a phage coat protein with only an amber stop codon intervening.

FIG. 5 shows how phage displayed antibodies, A, enter the flow chamber with rate constant Ã where their free concentration is A₁. There they can interact with peptide P, and recycle with rate constant ã. The antibody peptide forward and reverse rate constants k₁ and k₋₁ depend on the antibody combining site and peptide sequence.

FIG. 6 shows an example how phage and peptides are separated so that the ordering on the magnet preserves the ordering on the chip. The phage are dropped onto microtiter wells where they infect E. Coli. At the end of the process, each phage antibody can be associated with the mRNA encoding the peptide with which the antibody reacts.

FIG. 7 shows an example of magnetic separation of phage-peptide complexes. Biotin via covalently coupled to a phage coat protein. Streptavidin molecules, which coat the magnetic beads, bind biotin with high affinity. The complexes are lifted off each pixel in parallel, and the phage are deposited in microtiter wells containing E. Coli.

### DETAILED DESCRIPTION OF THE INVENTION

We have now discovered a high throughput method for producing large numbers of antibodies. The method uses microarrays of peptides which are used to screen large, combinatorially generated libraries of antibodies for specific binders. The invention chooses the peptides so that antibodies that bind to them, will also bind to them when they are a part of the protein. In this way a large library of antibodies against expressed proteins is obtained.

Additionally, we have discovered a method for the determination of proteins expressed by a given cell or tissue. The method utilizes microarrays of peptides, wherein one or more of the peptides are encoded by a coding region of the genome. Preferably, the peptides cover at least part of the coding regions that are of interest. For example, peptides from a family of proteins such as chemokine receptors, G-coupled protein receptors, a family of related proteins such as tumor associated antigens, oncogene products, etc. Alternatively the antibodies from these systems can first be solubilized using well known methods, and arrayed directly. Preferably, the chosen peptide contains an antigenic epitope. More preferably, the peptide has an epitope that approximates the wild type conformation of the protein. The arrays are then used to screen an antibody library such as a large, combinatorially generated library of antibodies that specifically bind to the peptides. Preferably, the antibodies bind to the peptides in a conformation in approximately their native state (i.e. when they are part of the protein). In this way, a large library of antibodies that will bind specific native proteins is obtained. These antibodies can be for any species whose genome is known for any desired group of proteins. The antibodies can then be expressed by known means such as simple bacterial amplification. The antibodies are arrayed on a substrate.

The term “antibody library” refers to a random library of antibody binding sites displayed on the surface of phage particles, plasmids, modified viruses, or bacteria as fusion coat proteins, for example.

The term “antibody array” refers to an ordered arrangement of antibodies, that specifically bind to peptide microarrays, on a substrate such as a glass, nylon, or a bead, such as SPA beads which is based on either yttrium silicate (YSi) which has scintillant properties by virtue of cerium ions within the crystal lattice, or polyvinyltoluene (PVT) which acts as a solid solvent for anthrancine (DPA) (Amersham Biosciences, Piscataway, N.J.).

The antibodies are arranged on the flat or spherical substrate referred hereto as a “chip” so that there are preferably at least one or more different antibodies, more preferably at least about 50 antibodies, still more preferably at least about 100 antibodies, and most preferably at least about 1,000 antibodies, on a 1 cm² substrate surface. The maximum number of antibodies on a substrate is unlimited, but can be at least about 100,000 antibodies.

The term “peptide microarray” refers to a microarray of peptides, wherein one or more of the peptides are from a coding region of the genome. Preferably, the peptides cover at least the coding regions that are of interest and contain an antigenic epitope. More preferably the peptide has an epitope that approximates the wild type conformation of the protein of interest.

A “plurality” refers preferably to a group of at least two or more members, more preferably to a group of at least about 100, and even more preferably to a group of at least about 1,000, members. The maximum number of members is unlimited, but preferably about 100,000 members.

The array can be made of any conventional substrate. Moreover, the array can be in any shape that can be read, including rectangular and spheroid. Preferred substrates are any suitable rigid or semi-rigid support including membranes, filter, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the peptides and/or antibodies are bound. Preferably, the substrates are optically transparent. Any type of substrate will be a suitable “chip” as long as the antibodies can be used as bait to fish for expressed proteins in a sample, such as a cell of interest.

The sample can be any sample obtained from any biological source, for example, blood, urine, saliva, phlegm, gastric juices, etc., cultured cells, tissue biopsies, or other tissue preparations.

Such antibody arrays can be used to screen a biological sample of interest. The proteins in the sample that bind to the array can be readily determined by a range of known means based upon this disclosure. For example, the target proteins and the antibodies may be labeled with one or more labeling moieties to allow detection of both protein-antibody complexes and by comparison the lack of such a complex in the comparison sample. The labeling moieties can include compositions that can be detected by photochemical, spectroscopic, biochemical, immunochemical, chemical, optical, electrical, bioelectronic, etc. means. Labeling moieties include chemiluminescent compounds, radioisotopes, labeled compounds, spectroscopic markers such as fluorescent molecules, magnetic labels, mass spectrometry tags, electron transfer donors and/or acceptors, etc.

By comparing the level of expression as measured by the changes in binding in, for example, the same type of tissue at different developmental stages, or in malignant vs. non-malignant or diseased vs. non-diseased cells, one can rapidly identify those proteins whose expression varies. The term “same type of tissue” and “similar tissue” are used interchangeably and mean generally tissue of a particular type such as, for example, kidney, heart, liver, brain, retina, bone and blood or particular fractions thereof, such as kidney glomeruli, heart valves, brain cortex, or white blood cells. It is also meant to describe tissue from the same organism such, for example human, mouse, or drosophila. Additionally, same or similar type of tissue means cell cultures established from such tissues or organisms.

Consequently, these arrays can be used for a wide range of purposes. For example, to determine proteins that are differentially expressed in related or different cells. For instance, malignant cells versus non-malignant cells, diseased cells versus normal, cells in a pregnant woman versus non-pregnant, menopausal versus non-menopausal, stem cells versus nerve cells, etc. The antibody arrays of the present invention can also be employed in numerous applications including diagnostics, prognostics and treatment regimens, drug discovery and development, toxicological and carcinogenicity studies, forensics, pharmacogenomics and the like, as explained more fully below. The present invention utilizes antibodies that are organized in an ordered fashion so that each antibody is present at a specified location on a two dimensional substrate. Because the antibodies are at specified locations on the substrate, the association between the antibody and the protein that it binds is known. This association is subsequently interpreted in terms of expression levels of particular proteins and, therefore, can be correlated with a particular disease or condition, or treatment.

The antibody arrays of the present invention can be applied to large scale genetic or gene expression analysis of a large number of target proteins. The arrays can also be used in the diagnosis of diseases and in the monitoring of treatments where altered expression of genes coding for proteins associated with cell proliferation or receptors cause disease, such as cancer, immunopathology, neuropathology, and the like. Further, the arrays can be employed to investigate an individual's predisposition to a disease, such as cancer, immunopathology, or a neuropathology. Furthermore, the arrays of the invention can be employed to investigate cellular responses to infection, drug treatment, and the like.

The present invention provides for an expression profile that can be used to detect changes in the expression of proteins implicated in disease. These proteins include proteins whose altered expression is correlated with cancer, immunopathology, apoptosis and the like.

The present invention yields expression profiles which comprise a plurality of antibody arrays and a plurality of detectable proteins. The antibody arrays are formed by screening an antibody library created by any one of the known display technologies (such as phage particles, plasmids, modified viruses, or bacteria as fusions to a coat protein) with peptide microarrays, wherein the peptides contain antigenic epitopes that approximates the wild type conformation of the proteins of interest. The antibody arrays are then used to screen a biological sample. The proteins that bind to the arrays can then be determined. The expression profiles obtained provide “snapshots” that show unique expression patterns characteristic of a disease or condition.

The present invention further provides a method for determining interactions between and among proteins, other molecules, and various organelles in order to determine numerous cellular functions such as proliferation, differentiation, gene expression, and cytoskeletal organization. The pattern of expressed proteins is an important marker for the state of the cell. The antibody arrays of the present invention are instrumental in associating proteins with their targets. Thus, using the antibody arrays, all expressed proteins are collected. Then, the genes for these proteins are amplified via standard PCR technology. Afterwards, a phage library is created to bind to targets in a manner fully analogous to the way antibody arrays were used. The genes for these targets are subsequently identified, amplified and used to bind their targets, and so on. In this way, a regulatory map of the cell under well-defined conditions is constructed.

Determination of phosphorylated proteins can be easily accomplished using antibodies directed against phosphotyrosines, for example. The state of methylation of proteins can be similarly determined. Any cell network, no matter how completely determined, will characterize the cell only under a well-defined set of conditions. Without wishing to be bound by theory, it can be expected that the changes in environment, in ligands impinging on the cell surface, will modulate the relative abundance of proteins in the network, change the expressed protein profile, and will even modulate cell network topology. Thus, a perturbation approach would provide valuable insight. The approach comprises first determining a reference network for a given set of conditions, and then systematically varying the concentration of a ligand specific for a particular key receptor from complete absence of the ligand to a concentration that gives receptor saturation, and constructing a network for each concentration employed.

The antibody arrays of the present invention can be used to monitor the progression of disease. Researchers can assess and catalog the differences in protein expression between healthy and diseased tissues or cells. By analyzing changes in patterns of protein expression, disease can be diagnosed at earlier stages before the patient is symptomatic. The invention can also be used to monitor the efficacy of treatment. For some treatments with known side effects, the antibody arrays can be employed to refine and customize the treatment regimen. A dosage can be established that causes a change in protein expression patterns indicative of successful treatment. Analogously, expression patterns associated with undesirable side effects can be avoided. This approach may be more sensitive and rapid than waiting for the patient to show inadequate improvement, or to manifest side effects, before altering the course of treatment.

Alternatively, animal models which mimic a disease, rather than patients, can be used to characterize expression profiles associated with a particular disease or condition. Hence, the protein expression data, as provided by the method of the present invention, may be useful in diagnosing and monitoring the course of disease in a patient, in determining gene targets for intervention, and in testing novel treatment regimens.

The expression of certain proteins is known to be associated with cell proliferation or receptors closely associated with cancers. Therefore, the antibody arrays and protein expression profiles of the present invention can be useful to diagnose, for example, a cancer such as, but not limited to adenocarcinoma, leukemia, lymphoma, melanoma, myeloma, sarcoma and teratocarcinoma, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid and uterus.

Proteins associated with cell proliferation may act directly as inhibitors or as stimulators of cell proliferation, growth, attachment, angiogenesis, and apoptosis, or indirectly by modulating the expression of transcription, transcription factors, matrix and adhesion molecules, and cell cycle regulators. In addition, cell proliferation molecules may act as ligands or ligand cofactors for receptors which modulate cell growth and proliferation. These molecules may be identified by sequence homology to molecules whose function has been characterized, and by the identification of their conserved domains. Proteins associated with cell proliferation may be characterized using programs such as BLAST or PRINTS. The characterized, conserved regions of proteins associated with cell proliferation and receptors may be used as probe sequences.

Receptor sequences are recognized by one or more hydrophobic transmembrane regions, cysteine disulfide bridges between extracellular loops, an extracellular N-terminus, and a cytoplasmic C-terminus. For example, in G protein-coupled receptors (GPCRs), the N-terminus interacts with ligands, the disulfide bridge interacts with agonists and antagonists, the second cytoplasmic loop has a conserved, acidic-Arg-aromatic triplet which may interact with the G proteins, and the large third intracellular loop interacts with G proteins to activate second messengers such as cyclic AMP, phospholipase C, inositol triphosphate, or ion channel proteins (Watson and Arkinstall (1994). The G-protein Linked Receptor Facts Book, Academic Press, San Diego Calif.). Other exemplary classes of receptors such as the tetraspanins (Maecker et al. (1997) FASEB J. 11:428-442), calcium dependent receptors (Speiss (1990) Biochem. 29:10009-18) and the single transmembrane receptors may be similarly characterized relative to their intracellular and extracellular domains, known motifs, and interactions with other molecules.

Furthermore, the expression of proteins associated with cell proliferation or receptors is also closely associated with the immune response. Therefore, the antibody arrays of the present invention can be used to diagnose immunopathologies including, but not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, and protozoal infections; and trauma.

One embodiment of the invention is a high throughput process for making one or more antibodies per protein, for a desired set of proteins encoded by a genome. The antibody arrays can then be used to assess how an expressed protein profile changes as the state of a cell changes or to compare profiles of different cells. Briefly, making an array for such an embodiment involves the following steps.

There are two alternative procedures for selecting peptides. One is to produce antibodies against continuous surface epitopes (typically 8-10 long) on a native protein, for example. This is done by exploiting the well known observation that antibodies elicited against a segment cleaved from a protein, will also react with the same segment in the native protein, if that segment is on the surface of the protein. If the crystal structure, or even the fold family, is known, picking the surface segments will not be difficult. If only the sequence is known some appropriate function of hydrophilicity must be calculated for each segment of the protein and a decision made about its location using (for example) discriminant analysis or its modern incarnation, support vector machines. Alternatively, every possible segment of the array can be synthesized, albeit with somewhat more labor. This exhaustive search, assures that every possible continuous surface epitope has been considered. There is another advantage to an exhaustive search. If the cell lysate is digested, interior segments become exposed. The exhaustive search will return antibodies against these segments, hence, almost all possible epitopes can be used, rather than just those on the surface as has been done traditionally in immunology.

Synthesize an array of peptides on a suitable substrate. For example, glass and nylon are preferred embodiments of the substrate. The glass or nylon chip size can be approximately 5 cm². The number of different peptide sequences can be 10, 50, 100, 1,000, 10,000 or 100,000. For instance, on the order of 100,000. The number of copies of each sequence is preferably 1-10 million.

The peptides can be made by a modification of standard chemistry for solid phase synthesis (2, 3). At each round of synthesis, the desired amino acid can be covalently coupled to oligopeptides at specified locations (pixels) on the chip by optically removing photolabile blocking groups terminating the oligos at those pixels, and then adding the desired amino acid or other known technique based upon the present disclosure. Removal of blocking groups at other pixels is preferably prevented by overlaying a physical mask which leaves only the desired pixels exposed to light. Thus, the synthesis of all oligopeptides N long would require 20N masking steps. Such a process is expensive. However, one can use an alternative, virtual masking, process that has been successfully employed for solid state oligonucleotide synthesis (4). It uses an array of micromirrors, each 16μ² and individually adjustable, to focus light on the desired set of pixels. This reduces the problem of changing the type or configuration of oligopeptides on the chip from having to design a new set of physical masks, to changing a few lines of code.

One can use any one of several display technologies to form a random library of antibody binding sites. One embodiment would be to display the sites on the surface of phage particles, plasmids, modified viruses, or bacteria as fusions to a coat protein, e.g. P3. Methods for creating such libraries are well known, see for example, Hoogenboom et al. (5).

The peptide microarray is then used to screen the antibody library, such as phage displayed antibodies, for those antibodies that bind specifically and with good affinity (>10⁶ M⁻¹).

Suitable separation technology known in the art are used based upon the present disclosure to purify the phage. The preferred embodiment is a variant of magnetic separation, as described below.

The antibodies selected are amplified by known techniques. For example amplifying the phage by infecting cells, such as E. coli.

The antibodies, such as phage are arrayed on a two dimensional surface so that the association between the antibody and the protein that it binds is known.

Neuronal processes are also affected by the expression of proteins associated with cell proliferation or receptors. Thus, the antibody arrays of the present invention can be used to diagnose neuropathologies including, but not limited to, akathisia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, neurofibromatosis, Parkinson's disease, paranoid psychoses, schizophrenia, and Tourette's disorder.

Also, researchers can use the antibody arrays of the present invention to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to determine the molecular mode of action of a drug.

It is understood that this invention is not limited to the particular methodology, protocols, and reagents described, as these may vary. It is also understood that the terminology used herein is for the purpose of describing particular embodiments only and is not intended to limit the scope of the present invention which will be limited only by the appended claims. The examples below are provided to illustrate the subject invention and are not included for the purpose of limiting the invention.

### EXAMPLES

Array Fabricator

The synthesis of all possible peptides of length N generally requires N 20-step rounds of chemistry and therefore a total of 20N steps in all. Each step adds one of the twenty amino acids to the growing chain, so that each round increments every chain by an amino acid. The growth step consists of using optical masks to selectively photodeprotect the oligo end groups in a selected number of pixels, and then flooding the chip with the desired blocked peptide.

The synthesis of all oligopeptide sequences N long, therefore, involves 20N physical masks. Although all sequences of a given length will generally not be needed, physical masking is nonetheless expensive and cumbersome.

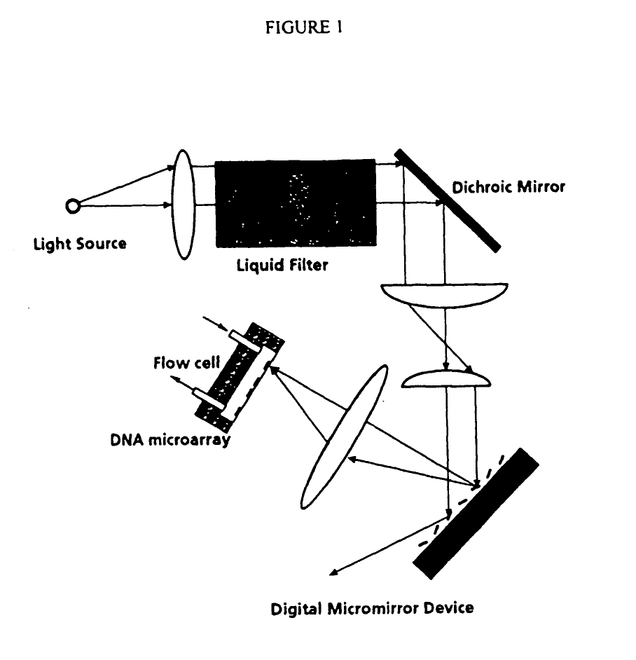

A recently developed alternative to physical masking uses an adaptable lens to focus UV light on specified pixels (4), thus selectively deblocking photolabile groups, while blocked groups remain in place at non illuminated pixels (FIG. 1). This allows polymerization of user determined amino acids at preprogrammed locations. Such virtual masking is rapid, inexpensive and automatable. Virtual masking has recently been applied to oligonucleotide synthesis.

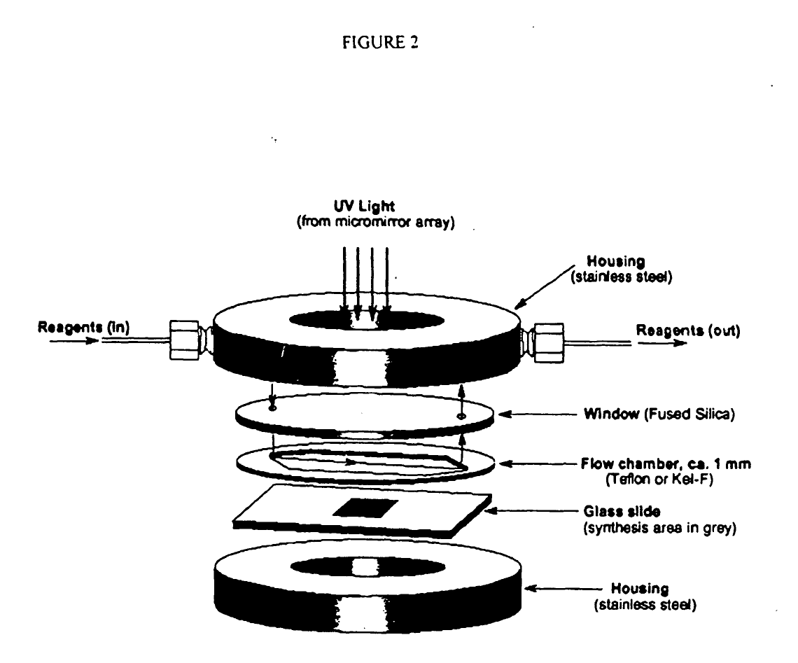

A complete array system requires (1) a digital micromirror assembly capable of being programmed to deliver UV light to a specific pixel; (2) a flow cell that contains the glass substrate (ca. 25 mm×25 mm), for example, shown in FIG. 2; and (3) a device for delivering reagents to the flow cell.

Selecting Peptides that Mimic Antigenic Sites in Native Proteins

In a preferred embodiment, sequences are chosen subject to the constraint that they be on the surface (solvent exposed) of the protein, otherwise antibodies produced against them would not be able to recognize the native protein, see, e.g. references 6-10. Preferably, such antibodies typically have affinities for the native sequences, 1-2 orders of magnitude lower than for the peptides used to select them, and are in the range of 10⁵-10⁶ M⁻¹. Immunological literature on the subject of eliciting antibodies cross-reactive with peptide in its free and native states spans some 25 years, e.g. (11, 12) (13, 14). The main requirement is that the sequence be hydrophilic, because it must be a protein surface sequence and therefore hydrated in the native state. The requirement of hydrophilicity is frequently supplemented with additional requirements; e.g. peptides encoded at exon/intron boundaries have a much higher probability than other sequences to be at boundaries between protein domains, and therefore solvent exposed. Similarly, amino terminal sequences tend to be solvent exposed. A suite of Bioinformatics algorithms can be used to select such peptides, and in a way that minimizes cross reactivity. For example knowledge of, or the ability to predict, exon/intron boundaries (15-17) adds to the ability to identify them when they are not known experimentally.

Synthesis of Ordered Oligomer Arrays Using Virtual Masking

Since the first demonstration nearly 10 years ago by Fodor et al. (18), at Affymax (now Affymetrix), of the principle of “light-directed, spatially addressable parallel chemical synthesis,” i.e., “synthesis on a chip,” there have been many advances in microarray technology. Although Fodor's original work described synthesis of peptide arrays, subsequent efforts have focused primarily on oligonucleotide arrays. Nevertheless, the technology for making peptide arrays exists and much of what has been learned about oligonucleotide arrays can be applied to peptides.

One of the problems with making arrays is the need for large numbers of photolithographic masks that permit selective deblocking of protected oligomers using UV light. The problem is severe in oligonucleotide synthesis where one needs four masks (corresponding to the four nucleotide bases) per synthetic cycle, but is much worse with peptides, where standard procedures would require 20 masks per cycle. To avoid this problem, we can use “maskless” microarray fabrication using anticromirror array such as described by (4).

The first step in the process of the present invention, as illustrated in FIG. 2, is derivatization of a glass surface with an appropriate alkoxysilane to give a surface coated with amino groups, each of which bears a photolabile protecting group. Specific areas (pixels) on the surface are deprotected by irradiation with UV light, which is directed to these areas by the micromirror assembly, and all the exposed amino groups are then acylated by an amino acid containing a photolabile protective group. In 19 subsequent steps, all of the remaining pixels are deprotected and acylated with the 19 remaining amino acids. This marks the end of the first synthetic cycle. The process is repeated until peptides of the desired length are obtained.

Derivatization of Glass Surface and Peptide Synthesis Chemistry

The preferred reagent for introduction of functionality onto glass surfaces for many years has been aminopropyltriethoxysilane and derivatives thereof. This reagent was introduced into protein sequencing nearly 30 years ago (19) and is currently widely used in the microarray fabrication of peptide and oligonucleotide libraries (4, 20, 21). In the case of DNA array synthesis, derivatives incorporating the hydroxybutyryl (21) or oligoethylene glycol (3, 22) moieties are often employed, but these are not appropriate for peptide synthesis because they contain a terminal hydroxyl, rather than amino group needed for peptide derivatization.

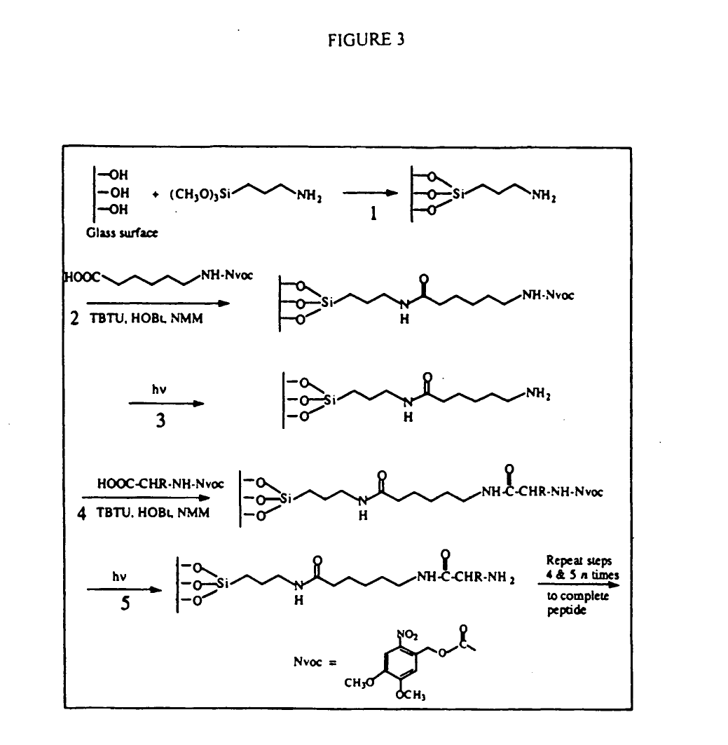

One embodiment of the present invention adapts the procedure of (20), namely silylyation with a 1:10 mixture of aminopropyltiiethoxysilane: methyltriethoxysilane (the latter added to reduce the density of amino groups by a factor of 10, followed by the addition of an aminocaproic acid linker containing the photolabile N-α-6-nitroveratyloxycarbonyl (Nvoc) group (FIG. 3). Activation during coupling steps can be done, preferably, using TBTU, a standard activating agent in peptide synthesis.

In another embodiment of the present invention, an aminocaproic acid linker with a longer or more hydrophilic (e.g., polyethylene glycol) linker can be substituted, if appropriate. Thus, in one embodiment of the invention, peptides of preferably 5-20mer (i.e., N=5-20), more preferably, 8-10mer peptides are synthesized, as epitope mapping studies (23) indicate that typical epitopes recognized by antibodies contain only about 6 amino acids. Because the number of different peptide sequences on a chip will be no more than several hundred thousand, only a very small fraction of all possible sixmers will be synthesized.

Protection and Deprotection of Amino Acids

Another aspect of the invention teaches how to selectively deprotect small, defined areas (pixels) on the glass surface. Deprotection thus requires efficient chemistry and engineering (i.e., the micromirror technology discussed earlier). Photolabile protective groups were first introduced by (24) and subsequently many variants have been described (25), most of which incorporate a 2-nitrobenzyl group.

Preferably, the N-α6-nitroveratyloxycarbonyl (Nvoc) group is used (similar to the one used successfully for peptide array synthesis (18)) and certain of the Nvoc amino acids are available commercially (from Peptides International, Inc., Louisville, Ky.); other Nvoc amino acids known in the art can also be synthesized. In another embodiment, the photolabile protecting groups such as the 2-(2-nitrophenyl)-propyloxycarbonyl (NPPOC) or α-methyl-2-nitropoiperonyl-oxycarbonyl (MeNPOC) groups described by (26) for oligonucleotide synthesis can be used. Any alternative derivative should be chosen with care, however, because it entails synthesis of an entire set of 20 amino acid derivatives. Preferably, Nvoc groups are removed by irradiation at ≧365 nm (20). Low wavelength light should be avoided to prevent destruction of certain amino acids, such as tryptophan.

It is an important aspect of the present invention that the length of time required to deprotect amino groups on a pixel be optimal. Among the preferred embodiments is the strategy of (21) for DNA arrays. The maskless array synthesizer (MAS) (4) is programmed to irradiate specific pixels or groups of pixels for varying periods of time, generating a gradient of partially to fully deprotected pixels. The glass substrate is then treated with any fluorescent reagent, preferably, fluorescein isothiocyanate (FrFC), and then visualized under the UV light. In such a way, the minimum time required for complete removal of the Nvoc (or any other) group can be determined. In the case of the Nvoc group, special attention should be given to the formation of photo byproducts that can act as an internal light masking agents (quencher) (27) thereby lowering the photochemical deprotection reaction. This can be avoided by flowing solvent through the flow cell of the MAS during photolysis to flush away by-products.

Display Libraries

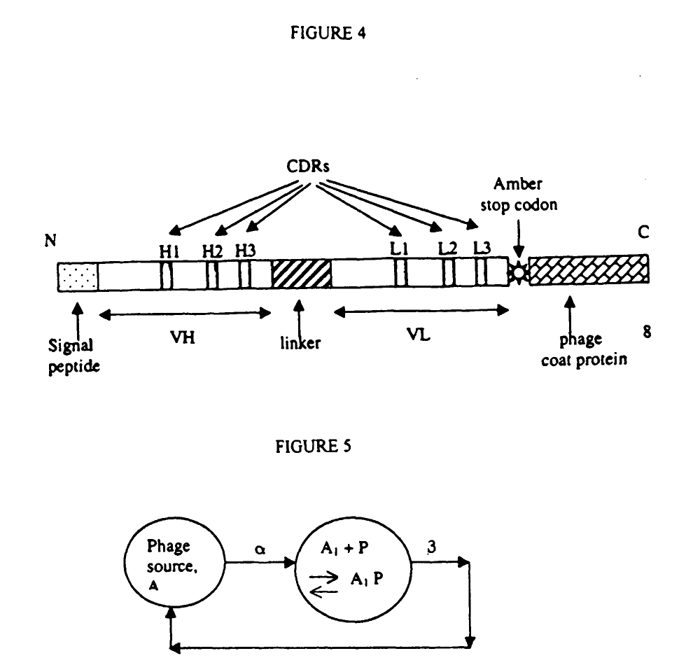

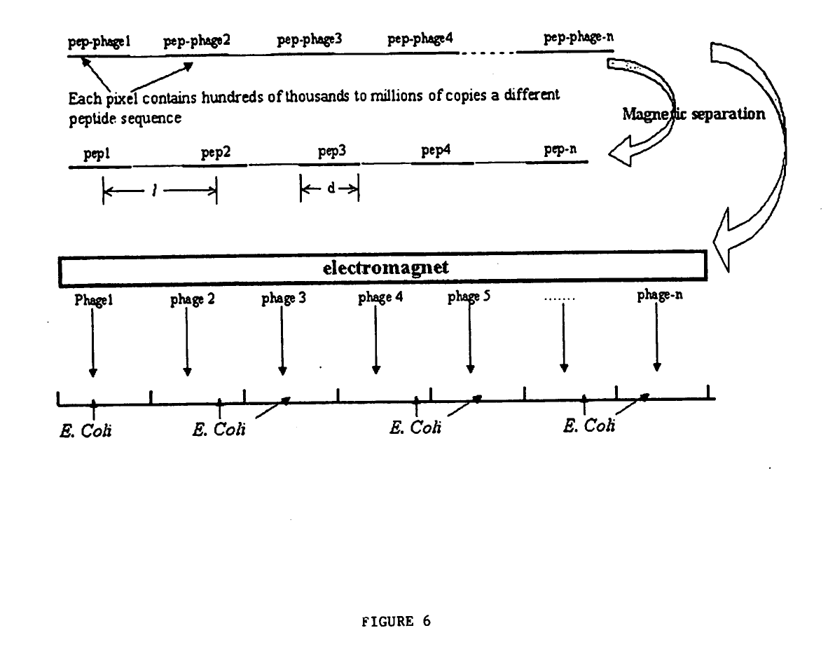

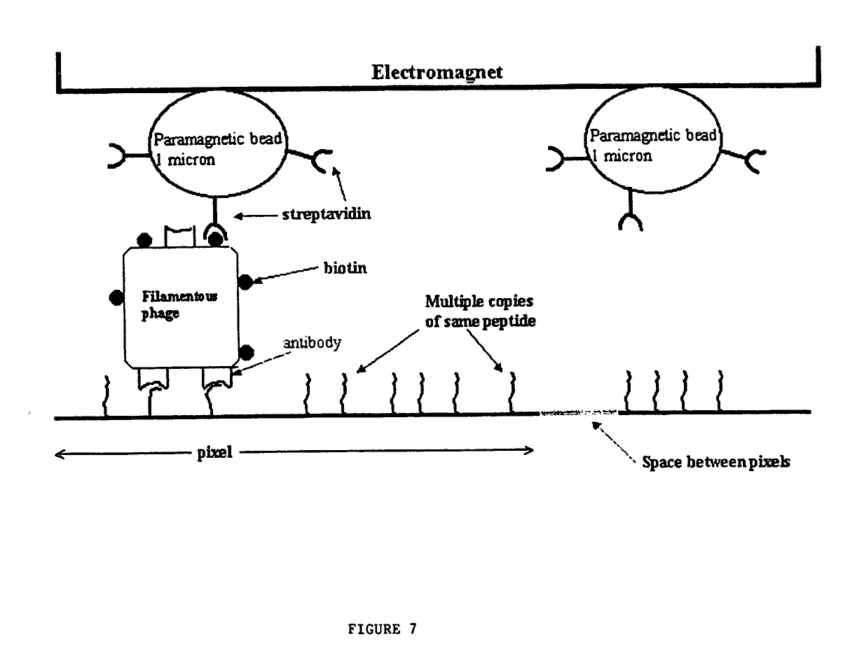

In one of the embodiments of the present invention, the genes encoding the amino terminal heavy (H) and light (L) chain immunoglobulins (Ig) domains, which comprise antibody combining sites, can be linked to form a single polypeptide chain and displayed as fusion surface proteins of either phage, plasmids, modified viruses, or bacteria (FIG. 4). A number of other embodiments are possible, e.g., using ribosomal display technology.

Briefly, for example, a phage-display library can be formed by reproducing phage in a strain of E. coli that ignores the amber stop codon thus producing fusion coat proteins. The resulting phage can, if necessary, be inserted into a bacterial strain that recognizes the stop signal, facilitating purification of the antibody.

In a typical combinatorial antibody library, 2 to 6 complementarity determining regions (CDRS) are randomized. A master phagemid is first constructed with H3 and L3 sequences that are known to facilitate the folding of the resulting scFv. Unique restriction sites terminate the framework sequences that are adjacent to the CDRS. These enable the substitution of subsequent H3 and L3 fragments with random sequences.

Randomized H3 and L3 sequences are generated via direct oligonucleotide synthesis. These are obtained during synthesis simply by using a mixture of nucleotide triphosphates (NTPs), rather than a single type of NTP, for one or more of the nucleotides of the central codon. NTPs will be selected randomly in accordance with their frequencies in the mixture, resulting in H3 and L3 with different sequences.

Direct synthesis of random CDRs can be difficult to control. However, the method of trinucleotide cassette mutagenesis generates a high quality randomized library because naturally occurring diversity is covered, both in terms of length and amino acid composition. A recently developed method that controls the specific amino acid composition at each position of the CDRs begins with the synthesis of 20 trinucleotide phosphoramidites. The appropriate stoichiometric amounts of phosphoramidites are then mixed and coupling is performed to yield longer oligonucleotides.

Once the master phagemid and the H3 and L3 cassette libraries are ready, they are cut with four unique restriction enzymes and ligated to form a phagemid library. After phage display, the phages with high-affinity scFv are picked out and the sequence of the scFv is easily determined using PCR with framework specific primers. If one round of selection does not produce high enough affinity, then DNA shuffling of the moderately binding clones can be used to further evolve the library.

Flow Chamber

Phage-peptide mixing, unlike hybridization of oligonucleotides, does not occur readily by diffusion. The size of the phage requires a flow chamber that mediates active mixing by transport. The relationship between the flow rate and time scales set by binding kinetics is crucial in phage-peptide mixing. The full analysis requires considering coupled diffusion reaction transport equations, but a compartmental model, as illustrated in FIG. 5, which holds when the flow rate is slow compared to the rate of peptide-phage binding provides an insight. Because the source and substrate are both heterogeneous, a superposition of such models is preferred.

The phage current entering the chamber (αP) will generally be different than the current leaving (βP₁), but rate constants α and β should be the same because the fluid is incompressible. When the rate constants α and β are set equal, the rate limiting time constant for system equilibration is
τ¹=−α+[α−βτ₁⁻¹+κ₁)]^{1/2}
where
α[2 β+τ₁⁻¹]/2
and the chemical reaction time scale is set by
ξ⁻¹=κ₁P+κ₁ (assuming peptide is not depleted by binding phage).

The result indicates that the rate at which equilibrium is approached increases as flow rate increases. This can in fact hold only if the flow rate is comparable to or less than the forward reaction rate κ₁P. The actual optimum can be found by performing a full analysis, including non-linearities.

Chemical reaction varies from pixel to pixel, because it depends on sequence. However, most of the variation is in the reverse rate constant, reflecting variations in binding energies (28). Therefore, the optimum flow rate is in the vicinity of κ₁P.

Typical peptide densities are preferably in the vicinity of 10¹⁰-10¹² cm⁻². Thus, for example, for a typical peptide of 30 A long, the concentration should be in the range of 5×10⁻⁵-5×10⁻³M. Forward rate constant for soluble antigen antibody interactions is preferably in the range of 10⁷ (sec-M)⁻¹, about two orders of magnitude below the Smoluchowski limit. For antibodies on a phage, the rate constant would be lower. Consequently, binding rates are preferred to be about 10⁴ sec⁻¹. While not wishing to be bound by theory, it is possible to have a very high flow rate without surpassing an optimum set by the chemical reaction.

Furthermore, the above model indicates that the concentration of phage bound at equilibrium is independent of the flow rate. The actual amount of phage bound, however, may depend upon peptide sequence. The highest affinities attainable by single site antibody attachment, without any special affinity maturation strategy, are preferably of order 10⁶-10⁷ M⁻¹. At planned peptide concentrations almost all antibodies are bound. It is preferable that the concentration which does not deplete peptides, such as 10⁷ phage/cm², be used.

Molecular Recognition

The following describes preferred physical conditions that are necessary to optimize the binding of phage to peptides.

Densities

The relevant quantities for the embodiment of the present invention are: (1) the number of pixels per slide which determines the number of different antibodies that can identified; (2) the spacing between pixels which is important for some separation procedures as further explained below; (3) the density of peptides within a pixel which determines the nature of binding, e.g., monovalent vs. multivalent; and (4) the overall size of the slide, which determines the quantity of material that must be used and therefore affects cost.

### Example 1

For a square chip with s pixels in each direction, the pixel dimension is d, and the center-to-center distance between pixels is, the characteristic dimension of a phage head is w and w 10⁻⁵ cm. On average, each head would have two P3 proteins and therefore display two antibodies. The area of a chip with N2 pixels is
A=[(s−1)l+d]2.

When s=100, l=d, d=0.01 cm, and an average of 10,000 peptides/cm (1 million peptides per 0.01 cm² pixel), the mean spacing between peptides is 10⁻⁴ cm. Under these conditions adjacent peptides do not interact physically because even a fully extended peptide with 20 residues would only span 6×10⁻⁷ cm. Additionally, because the spacing between peptides is greater than the dimension of the phage head, it is unlikely that more than one antibody will be bound to the same phage, therefore, phage binding would be monovalent. Because affinities of an antibody for a peptide are usually low, multivalent attachment would be desirable. A density of 10¹⁰-10¹² peptides/cm² is preferred for multivalent attachment because it is sufficiently low to prevent physical interaction between adjacent peptides. These densities are exemplary averages over the entire surface, and therefore, it is likely that fluctuations in densities would reduce the amount of multivalent binding of phage per pixel.

Time Constraints

In the preferred embodiment of the present invention, phage must be separated from tens of thousands of pixels before it dissociates. In order to estimate the time constraints this imposes, the amount of binding that can be expected under a given set of conditions and the amount remaining as a function of time after irrelevant phage is rinsed off the chip must be known. In addition, the materials, methods and examples are illustrative only and not intended to be limiting.

### Example 2

Let T be the size of the antibody display library, i.e. the number of distinct antibody binding sites (typically billions). It is generally expected that more than one of the T distinct antibodies will recognize a particular peptide sequence. Consider a typical peptide sequence at concentration L. Let cⱼ be the total concentration of phage available to bind it with affinity Kⱼ; let bⱼ be the concentration of these antibodies that are bound. Then,

$${b}_{j} = \frac{{K}_{j} {c}_{j} L}{1 + {K}_{j} L}$$

and define C_{T} as the total phage concentration:

$$\begin{matrix}\sum {b}_{j} = \sum \frac{{K}_{j} {c}_{j} L}{1 + {K}_{j} L} L \sum [ {K}_{j} {c}_{j} - {K}_{j}^{2} {c}_{j} + {K}_{j}^{3} {L}^{2} - … ] \\ = {C}_{T} [ < K > L - < {K}^{2} > {L}^{2} + < {K}^{3} > {L}^{3} - … ]\end{matrix}$$

Let the solution layered on the slide contain on average n copies of each of the T phages; i.e. the total number of phage is nT, and these are distributed throughout a volume v=[(s−1)l+d]2h, where h is the height of fluid on the slide. Then C_{T}=nT/v. In addition, if [symbol] is the density of peptides, then L=σ/h. To a first approximation, with l=d, the ratio of the concentration of bound antibodies to total peptide concentration is:

$$\frac{B}{L} = \frac{nT < K >}{{h ( ds )}^{2}}$$

For illustration purposes, if <K>=10⁶M⁻; T=10⁹; n=10,000; h=0. 1 cm; s=100 pixels/row; d=0.01 cm. Then, approximately 2% of the peptides will be bound by phage, or approximately 2000 phage per pixel.

Affinities this low are usually accompanied by rapid dissociation. Thus, using these numbers, at time t after rapidly rinsing away unbound phage, and taking a reverse rate constant of 0.1 sec⁻¹, the amount of specifically bound phage will be 2000 exp(−0.1 t). This does not allow adequate time for ordered removal and storage of specifically bound phage. A comparable analysis gives an equation for multivalent attachment. With 10¹⁰ peptides/cm², the rate of dissociation is decreased by 2-3 orders of magnitude, allowing adequate time for ordered removal of phage (good sensitivity), although some mixing with phage from adjacent pixels will still occur.

Phagemid Purification

Phage must be removed from each pixel in a way that preserves the association between the phage and the protein it recognizes. Since this needs to be done quickly, phage must be removed from all pixels simultaneously. We will achieve massively parallel purification by biotinylating the bound phage, and then using streptavidin coated magnetic beads to lift the phage from the slide. The lifting can be done in parallel by using an electromagnetic, which then deposits each group of phage in corresponding wells containing E Coli.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

The references cited below and incorporated throughout the application are incorporated herein by reference.

### REFERENCES

O'Farrell, P. H., High resolution two-dimensional electrophoresis of proteins, J Biol Chem, 250, 4007 (1975).
Lipshutz, R. J., Fodor, S. P., Gingeras, T. R., and Lockhart, D. J., High density synthetic oligonucleotide arrays, Nat Genet, 21, 20 (1999).
Southern, E., Mir, K., and Shchepinov, M. Molecular interactions on microarrays, Nat Genet, 21, 5 (1999).
Singh-Gasson, S., Green, R. D., Yue, Y., Nelson, C., Blattner, F., Sussman, M. R., and Cerrina, F., Maskless fabrication of light-directed oligonucleotide microarrays using a digital micromirror array [see comments], Nat Biotechnol, 17, 974 (1999).
Hoogenboom, H. R., de Bruine, A. P., Hufton, S. E., Hoet, R. M., Arends, J. W., and Roovers, R. C., Antibody phage display technology and its applications, Immunotechnology, 4, 1(1998).
Ayaki, M., Hashimoto, C., and Inui, Y., Characterization of a 34 kDa immunoreactive peptide with the anti protein kinase C antibody which can recognize part of the C4 region, Biochem Mol Biol Int, 41, 969 (1997).
Hama, T., and Maruyama, M., Development of an antibody against a 40,000 mol. wt brain injury-derived neurotrophic peptide-binding protein and identification of a 40,000 mol. wt brain injury-derived neurotrophic peptide-binding protein in hippocampal neurons, Neuroscience, 98, 567 (2000).
Ikushima, M., Yamada, F., Kawahashi, S., Okuyama, Y., and Matsui, K., Antibody response to OspC-I synthetic peptide derived from outer surface protein C of Borrelia burgdorferi in sera from Japanese forestry workers, Epidemiol Infect, 122,429 (1999).
Partidos, C. D., Ripley, J., Delmas, A., Obeid, 0. E., Denbury, A., and Steward, M. W., Fine specificity of the antibody response to a synthetic peptide from the fusion protein and protection against measles virus-induced encephalitis in a mouse model, J Gen Virol, 78, 3227 (1997).
Yang, H. W., Hemmi, H., Ikeda, H., Kato, K., and Tsuchida, Y., A polyclonal antibody against synthetic peptide conserved in N-Myc protein reacts with water-soluble recombinant N-Myc protein, Oncol Rep, 6,107 (1999).
Fae, B., Schechter, A. N., Sachs, D. H., and Anfinsen, C. B., An immunological approach to the conforinational equilibrium of staphylococcal nuclease, J Mol Biol, 92,497 (1975).
Berzofsky, J. A., and Schechter, A. N., The concepts of crossreactivity and specificity in immunology, Mol Immunol, 18, 751 (1981).
Sachs, D. H., Schechter, A. N., Eastlake, A., and Anfinsen, C. B., An immunologic approach to the confonnational equilibria of polypeptides, Proc Natl Acad Sci USA, 69, 3790 (1972).
Lando, G., Berzofsky, J. A., and Reichlin, M., Antigenic structure of sperm whale myoglobin. 1. Partition of specificities between antibodies reactive with peptides and native protein, J Immunot, 129, 206 (1982).
Novichkov, P. S., GelTand, M. S., and Mironov, A. A., [Prediction of the exonintron structure by comparing nucleotide sequences from various genomes], Mol Biol (Mosk), 34, 230 (2000).
Batzoglou, S., Pachter, L., Mesirov, J. P., Berger, B., and Lander, E. S., Human and mouse gene structure: comparative analysis and application to exon prediction, Genome Res, 10, 950 (2000).
Saxonov, S., Daizadeh, I., Fedorov, A., and Gilbert, W., EID: the Exon-Intron Database—an exhaustive database of protein-coding intron-containing genes, Nucleic Acids Res, 28, 185 (2000).
Fodor, S. P., Read, J. L., Pirrung, M. C., Stryer, L., Lu, A. T., and Solas, D., Light-directed, spatially addressable parallel chemical synthesis, Science, 251, 767 (1991).
Laursen, R. A., and Machleidt, W., Solid-phase methods in protein sequence analysis, Methods Biochem Anal, 26, 201 (1980).
Holmes, C. P., Adams, C. L., Kochersperger, L. M., Mortensen, R. G., and Aldwin, L. A., The use of light-directed combinatorial peptide synthesis in epitope mapping, Biopolymers Peptide Sci, 3 7, 199 (1995).
McGall, G. H., Barone, A. D., Diggehnann, M., Fodor, S. P. A., Gentalen, E., and Ngo, N., The efficiency of light-directed synthesis of DNA arrays on glass substrates, J. Am. Chem. Soc., 119,5081 (1997).
Elder, J. K., Johnson, M., Milner, N., Mir, K. U., Sohail, M., and Southern, E. M., Antisense oligonucleotide scanning arrays, in DNA Microarrays: A Practical Approach, Schena, M., Ed., pp. 77 (1999).
Wang, Z., Camey, W. P., and Laursen, R. A., Epitopic characterization of the human wild-type and mutant ras proteins using membrane-bound peptides, J Pept Res, 50,483 (1997).
Patchomik, A., Arnit, B., and Woodward, R. B., Photolabile,) J. Am. Chem. Soc, 92,6333(1970).
Pillai, V. N. R., Synthesis, Synthesis, 1, 1 (1980).
Beier, M. a. H., J. D, Production by quantitative photolithographic synthesis of individually quality checked DNA microartays, Nucleic Acids Res, 28, 11 (2000).
Ajayaghosh, A., and Pillai, V. N. R, Solid-phase synthesis and C-terminal amidation of peptides using a photolabile o-nitrobenzhydrylaminopolystryene support, Tetrahedron Lett, 36, 777 (1995).
Delisi, C., Hemolytic plaque inhibition: the physical chemical limits on its use as an affinity assay, J Immunol, 11 7, 2249 (1976).
   All references described herein are incorporated herein by reference.

1. A method for a fast high-throughput determination of proteins differentially expressed by a cell or a plurality of cells comprising:
(a) subjecting a first biological sample to a microarray containing at least 100 different antibodies;
(b) comparing the protein profiles of said first biological sample with a second biological sample; and
(c) identifying proteins that are differentially expressed in at least part of the cells of said biological samples.

2. The method of claim 1, wherein the first and second biological sample are from a similar tissue but differ from each other by developmental stage.

3. The method of claim 1, wherein the first and second biological sample are from a similar tissue but differ from each other by hormone expression.

4. The method of claim 1, wherein the first biological sample is from a normal or non-diseased tissue and the second biological sample is from a diseased-tissue or tissue suspected of being diseased.

5. The method of claim 1, wherein the first biological sample is from a normal or non-malignant tissue and the second biological sample is from a malignant tissue or a tissue suspected of being malignant.

6. The method of claim 1, wherein the first biological sample is from a normal or non-infected tissue and the second biological sample is from an infected tissue or from a tissue suspected of being infected.

7. The method of claim 1, wherein said microarray contains at least 1,000 antibodies.

8. The method of claim 1, wherein said microarray contains at least 10,000 antibodies.

9. The method of claim 1, wherein said microarray contains at least 100,000 antibodies.

10. A method of high-throughput synthesis of a plurality of antibodies comprising:
(a) synthesizing a peptide microarray on a suitable substrate using virtual masking;
(b) forming a random antibody library;
(c) screening the random antibody library with the peptide microarray and selecting antibodies that bind with a suitable affinity and specificity to said peptide microarray;
(d) purifying the selected antibodies;
(e) amplifying the purified antibodies; and
(d) binding the antibodies to a substrate thereby forming a microarray of antibodies suitable for high-throughput analysis.

11. The method of claim 10, wherein the substrate is glass or nylon.

12. The method of claim 10, wherein the microarray of antibodies contains at least 1,000 antibodies.

13. The method of claim 10, wherein the microarray of antibodies contains at least 10,000 antibodies.

14. The method of claim 10, wherein the microarray of antibodies contains at least 100,000 antibodies.

15. The method of claim 10, wherein the random antibody library is formed using a phage-display library.
